(19) United States
(12) Patent Application Publication
(10) Pub. No.: US 2019/0040460 A1
(43) Pub. Date: Feb. 7, 2019

(54) METHOD FOR THE PRODUCTION AND FORGERY-PROOF AUTHENTICATION OF A MEDICAL DEVICE BY USING A NUCLEIC ACID WITH KNOWN PARTIAL SEQUENCE

(71) Applicant: Fraunhofer-Gesellschaft zur Forderung der angewandten Forschung e.V., Munchen (DE)

(72) Inventors: Joachim Storsberg, Worrstadt (DE); Marina Volkert, Berlin (DE)

(21) Appl. No.: 16/044,824

(22) Filed: Jul. 25, 2018

(30) Foreign Application Priority Data
Jul. 28, 2017 (DE) 10 2017 007 181.0

(51) Int. Cl. C12Q 1/6869; A61L 27/20; A61L 27/18

(52) U.S. Cl. CPC C12Q 1/6869; A61F 2/12; A61L 27/18; A61L 27/20

(57) ABSTRACT

The invention relates to a method for the production and forgery-proof authentication of medical devices by using a nucleic acid having a known partial sequence, the method comprising the following steps:
(i) producing a medical device (MED) having a permanent marking (PM), wherein the medical device (MED) comprises a polymer (P) with the permanent marking (PM) and the permanent marking (PM) contains at least one nucleic acid (NUK) with a known partial sequence (TSEQ);
(ii) isolating the permanent marking (PM); and
(iii) authentication of the medical device (MED) by detection of the at least one known partial sequence (TSEQ).

The invention relates to a method of producing and forgery-proof authentication of a medical device by using a permanent marking comprising at least one nucleic acid having a known partial sequence.

The forgery-proof identification of valuables and security documents by the external application of a marking solution based on nucleic acids is known and described for example in WO 03/038000 A1.

Medical devices typically provide for the consumer a health risk and for the manufacturer a considerable financial risk because of potential damages or compensation claims. There is a great interest in designing medical devices in such a way that they are provided with forgery-proof security features with which the authenticity can be verified.

In connection with the forgery-proof marking of medical devices a number of difficulties arise which need not be taken into account in the forging proof marking of valuables and security documents.

The security features should be applicable as universally as possible and they should have a high protection against counterfeiting or forgery. The safety features should neither leak uncontrollably from the medical device nor adversely affect its properties.

A particular challenge is the forgery-proof marking of materials, which are further processed to a final product by third parties.

These and other objects are achieved by the present invention. The invention is described in the independent claims; advantageous embodiments are specified in the dependent claims.

### SUMMARY

A method of authenticating a medical device (MED) is described, comprising the following steps:
(i) producing a medical device (MED) with a permanent marking (PM), wherein the medical device (MED) comprises a polymer (P) with the permanent marking (PM) and the permanent marking (PM) contains at least one nucleic acid (NUK) with a known partial sequence (TSEQ);
(ii) isolating the permanent marking (PM); and
(iii) authentication of the medical device (MED) by detection of the at least one known partial sequence (TSEQ).

In one embodiment, the detection comprises the amplification of at least one known partial sequence (TSEQ) of the at least one nucleic acid (NUK).

In one embodiment, the detection comprises the sequencing of at least one known partial sequence (TSEQ) of the at least one nucleic acid (NUK).

In one embodiment, the detection comprises the hybridization of the at least one known partial sequence (TSEQ) of the at least one nucleic acid (NUK) with a detection nucleic acid.

In one embodiment, the authentication of the medical device (MED) by detection of the at least one known partial sequence (TSEQ) comprises the following steps:
(i) determining the concentration (EK) of the permanent marking (PM) in the polymer (P) of the medical device (MED) to be authenticated;
(ii) comparing the concentration (EK) of the permanent marking with the concentration (AK) of the permanent marking (PM) of the polymer (P) used to make the medical device (MED).

Also described is a method of producing a medical device (MED) with a permanent marking (PM) comprising the following steps:
(i) providing a polymer (P) with a permanent marking (PM), wherein the permanent marking (PM) contains at least one nucleic acid (NUK) with at least one known partial sequence (TSEQ); and
(ii) processing the polymer (P) into a medical device (MED).

In one embodiment the method of producing the medical device (MED) comprises the following steps of:
(i) providing at least one monomer (M);
(ii) providing a permanent marking (PM) containing at least one nucleic acid (NUK) with a known partial sequence (TSEQ);
(iii) distributing the permanent marking (PM) in the at least one monomer (M);
(iv) polymerizing the at least one monomer (M) to obtain a polymer (P) comprising the permanent marking (PM); and
(v) processing the polymer (P) into a medical device (MED).

In one embodiment, the method of producing the medical device (MED) comprises the following steps of:
(i) providing a polymer (P);
(ii) providing a permanent marking (PM);
(iii) distributing the permanent marking (PM) in the polymer (P); and
(iv) processing the polymer (P) into a medical device (MED).

In one embodiment, the method of producing the medical device (MED) comprises the following steps of:
(i) providing a polymer (P);
(ii) providing a solvent (LM);
(iii) dissolving the polymer (P) in the solvent (LM);
(iv) providing a permanent marking (PM);
(v) distributing the permanent marking (PM) in the dissolved polymer (P); and
(vii) processing the polymer (P) into a medical device (MED).

In the described methods, the polymer (P) contained in the medical device may be crosslinked.

In the described method the nucleic acid (NUK) with a known partial sequence (TSEQ) can be a deoxyribonucleic acid (DNA).

In the described process, the medical device (MED) is a medical device of class III.

In the described method in addition to the nucleic acid (NUK) with a known partial sequence (TSEQ) the permanent marking (PM) can contain at least one further nucleic acid which does not have the partial sequence (TSEQ).

A medical device (MED) with a permanent marking (PM) is described which comprises a polymer (P) with a permanent marking (PM), wherein the permanent marking (PM) contains at least one nucleic acid (NUK) with a known partial sequence (TSEQ).

The use of a permanent marking (PM) is described which comprises at least one nucleic acid (NUK) with a known partial sequence (TSEQ) for authenticating a medical device (MED).

### DETAILED DESCRIPTION

A method of authenticating a medical device (MED) is described, comprising the following steps:
(i) producing a medical device (MED) with a permanent marking (PM), wherein the medical device (MED) comprises a polymer (P) with the permanent marking (PM), and the permanent marking (PM) contains at least one nucleic acid (NUK) having a known partial sequence (TSEQ);
(ii) isolating the permanent marking (PM); and
(iii) authentication of the medical device (MED) by detection of the at least one known partial sequence (TSEQ) in the isolated at least one nucleic acid (NUK).

The medical device is authentic if the at least one known partial sequence (TSEQ) can be detected in the at least one nucleic acid (NUK) which is contained in the isolated permanent marking (PM).

The detection can be carried out by a method in which the at least one known partial sequence (TSEQ) contained in the permanent marking (PM) is initially amplified.

In this embodiment, the method of authenticating a medical device (MED) comprises the following steps:
(i) producing a medical device (MED) with a permanent marking (PM), wherein the medical device (MED) comprises a polymer (P) with the permanent marking (PM) and the permanent marking (PM) contains at least one nucleic acid (NUK) having a known partial sequence (TSEQ);
(ii) isolating the permanent marking (PM);
(iii) authentication of the medical device (MED) by detecting the at least one known partial sequence (TSEQ) in the at least one nucleic acid (NUK), wherein the detection comprises the amplification of the at least one known partial sequence (TSEQ) of at least one nucleic acid (NUK).

The medical device is authentic when the at least one known partial sequence (TSEQ) can be detected in the at least one nucleic acid (NUK) which is contained in the isolated permanent marking (PM) and was amplified.

But the proof can also directly take place without any amplification of the known partial sequence (TSEQ).

For example, it is possible to detect the at least one known partial sequence (TSEQ) by sequencing. For this purpose, the sequence of the at least one known partial sequence (TSEQ) of the at least one nucleic acid (NUK) contained in the isolated permanent marking (PM) is determined.

However, the detection by sequencing need not be done directly, but can also include the amplification of the known partial sequence (TSEQ) in addition to sequencing.

Accordingly, in one embodiment the method of authenticating a medical device (MED) includes the following steps:
(i) producing a medical device (MED) with a permanent marking (PM), wherein the medical device (MED) comprises a polymer (P) with the permanent marking (PM), and the permanent marking (PM) contains at least one nucleic acid (NUK) having a known partial sequence (TSEQ);
(ii) isolating the permanent marking (PM);
(iii) authentication of the medical device (MED) by detection of the at least one known partial sequence (TSEQ) in the at least one nucleic acid (NUK), wherein the detection comprises sequencing of the at least one known partial sequence (TSEQ) of the at least one nucleic acid (NUK).

It is also possible to detect the at least one known partial sequence (TSEQ) by hybridization with a ‘detection nucleic acid’ i.e. a nucleic acid for detection. For this purpose, the at least one known partial sequence (TSEQ) of the at least one nucleic acid (NUK) contained in the isolated permanent marking (PM) is hybridized with a detection nucleic acid which is at least partially complementary to the known partial sequence (TSEQ) of the nucleic acid (NUK). The detection by hybridization with a detection nucleic acid can be performed directly, but can comprise in addition to the hybridization with a detection nucleic acid the amplification of the known partial sequence (TSEQ).

Accordingly, in an embodiment the method of authentication of a medical device (MED) comprises the steps of:
(i) producing a medical device (MED) with a permanent marking (PM), wherein the medical device (MED) comprises a polymer (P) with the permanent marking (PM), and the permanent marking (PM) contains at least one nucleic acid (NUK) having a known partial sequence (TSEQ);
(ii) isolating the permanent marking (PM);
(iii) authentication of the medical device (MED) by detection of the at least one known partial sequence (TSEQ) in the at least one nucleic acid (NUK), wherein the detection is the hybridization of the at least one known partial sequence (TSEQ) of the at least one nucleic acid (NUK) with a detection nucleic acid.

The term “medical device (MED)” refers to an article or a substance used for medical therapeutic or diagnostic purposes for humans, where in contrast to drugs the intended main effect is primarily not pharmacologically, metabolically or immunologically, but usually physically or physicochemically, and is defined as follows:

Medical devices are all instruments, apparatuses, devices, substances or other objects which are used alone or interlinked, including those specifically intended by the manufacturer for diagnostic and/or therapeutic use, which are intended by the manufacturer for detection, prevention, monitoring, treatment or alleviation of human diseases; the detection, monitoring, treatment, alleviation or compensation of injury or disability; the examination, replacement or alteration of the anatomical structure or a physiological process; or the contraception and the intended main effect of which—in or on the human body—is achieved neither by pharmacological or immunological means nor in a metabolic manner, but which mode of action can be supported by such means.

The medical device (MED) can be selected from the group consisting of Class I, i.e. medical instruments, crutches, wheelchairs, hospital beds, support stockings, dressings, reusable surgical instruments, surgical textiles; Class IIa, i.e. dental materials, disinfectants, ultrasonic diagnostic equipment, disposable syringes, hearing aids, contact lenses, picture archiving and communication system (PACS), tracheal tubes, tooth crowns; Class IIb, i.e. anesthesia equipment, ventilation equipment, radiation equipment, blood bags, defibrillators, dialysis machines, condoms, contact lens cleaners, dental implants, cleaning disinfector; and Class III, i.e. cardiac catheter, artificial hip, knee or shoulder joints, stents, resorbable surgical sutures, intrauterine devices, breast implants, pacemakers, as specified in the Annex IX of the Directive 93/42/EEC.

In particular, the medical device (MED) may be selected from the group consisting of cardiac catheter, artificial hip, knee or shoulder joints, stents, resorbable surgical suture, intrauterine device, breast implant, and pacemaker.

In a preferred embodiment, the medical device (MED) is a medical device of Class III.

In a particularly preferred embodiment the medical device (MED) is a breast implant.

The method of manufacturing the medical device (MED) depends on the medical device itself. The corresponding methods are known in the art and are chosen as required.

A method of producing a medical device (MED) with a permanent marking (PM) comprising the following process steps:
(i) providing a polymer (P) with permanent marking (PM), wherein the permanent marking (PM) contains at least one nucleic acid (NUK) with a known partial sequence (TSEQ);
(ii) processing the polymer (P) into a medical device (MED).

The term “permanent marking” refers to a marking that can be used to perform a qualitative and/or quantitative authentication method, whereby the marking is preferably permanently associated with the medical device to be authenticated.

In other words, the permanent marking cannot be removed from the medical device to be authenticated without the use of further auxiliary means. In particular, the permanent marking of a medical device used on humans is essentially not released to humans. In particular, the permanent labeling of a medical device used on humans is not released to humans in pharmacologically and/or toxicologically relevant quantities.

The term “permanent” refers at least to the medical device (MED). In one embodiment, the term “permanent” also refers to the polymer (P) in addition to the medical device (MED). In other words, the permanent marking (PM) is permanently connected at least to the medical device (MED). In one embodiment, the permanent marking (PM) is also permanently attached to the polymer (P).

The fact that the marking contained in the polymer in the applied medical device is not released to humans can be achieved in different ways. If the polymer (P) used to manufacture the medical device (MED) which comprises the permanent marking (PM) is in direct contact with humans when applied to humans, the nature of the polymer itself results in that the marking is not substantially released to humans, in particular not in pharmacologically and/or toxicologically relevant amounts.

If the polymer (P) comprising the permanent marking (PM) which is used for the production of the medical device (MED) is not available for human use in direct contact with humans, then the nature of the polymer can also ensure that the permanent marking (PM) is not substantially delivered to the human, in particular not in pharmacologically and/or toxicologically relevant amounts. In such a case, however, also other components of the medical device (MED) having barrier properties against the permanent marking (PM) may ensure that the permanent marking (PM) of the medical device used on humans is substantially not released to the humans, in particular not in pharmacologically and/or toxicologically relevant amounts.

Suitable auxiliary means for removing the permanent marking (PM) from the medical device (MED) to be authenticated are e.g. solvents, reagents which cause a degradation of the medical device (MED) or a mechanical removal of the permanent marking from the medical device (MED) by a purposive external action.

The known methods, i.e. methods of producing a medical device (MED) with a permanent marking (PM), may need to be slightly modified to incorporate the permanent marking (PM) into the polymer (P) used.

Typically, such a modified method of making a medical device (MED) with a permanent marking (PM) comprises the following steps:
(i) providing at least one monomer (M);
(ii) providing a permanent marking (PM) comprising at least one nucleic acid (NUK) having a known partial sequence (TSEQ);
(iii) distributing the permanent marking (PM) in the at least one monomer (M);
(iv) polymerizing the at least one monomer (M) to obtain a polymer (P) comprising the permanent marking (PM);
(v) processing the polymer (P) into a medical device (MED).

The permanent marking (PM) can be distributed before and/or during the polymerization in the at least one monomer (M).

Alternatively, the at least one monomer (M) may react with a polymer (P) first and then permanent marking (PM) may be incorporated into the polymer after the polymerization.

Typically, such a method of producing a medical device (MED) with a permanent marking (PM) comprises the following steps:
(i) providing a polymer (P);
(ii) providing a permanent marking (PM);
(iii) distributing the permanent marking (PM) in the polymer (P);
(iv) processing the polymer (P) into a medical device (MED).

In this context it is possible, for example, to dissolve the polymer (P) in an appropriate solvent, to distribute the permanent marking (PM) in the dissolved polymer (P) and then to deposit or precipitate the polymer (P) from the solvent.

Typically, such a modified method for manufacturing a medical device (MED) with a permanent marking (PM) comprises the following steps:
(i) providing a polymer (P);
(ii) providing a solvent (LM);
(iii) dissolving the polymer (P) in the solvent (LM);
(iv) providing a permanent marking (PM);
(v) distributing the permanent marking (PM) in the dissolved polymer (P);
(vi) processing the polymer (P) into a medical device (MED).

Optionally, after distribution of the permanent marking (PM) in the dissolved polymer (P), the polymer (P) may be separated from the solvent (LM) and then it may be processed to a medical device (MED) In this case, the method for producing a medical device (MED) with a permanent marking (PM) comprises the step of separating the polymer (P) from the solvent (LM).

The conditions under which the process steps of dissolving the polymer (P), distributing the permanent marking (PM) and separating the polymer (P) are carried out depend on the nature of the polymer (P) and the solvent (LM) used. These are known to the person skilled in the art and are selected as needed.

Alternatively, it is possible to swell the polymer (P) in a suitable solvent (LM), so that the permanent marking (PM) is incorporated in the polymer (P). In this case, the permanent marking (PM) is preferably initially distributed in the swelling agent (QM), in particular dissolved or dispersed, and then the polymer (P) comprising the permanent marking (PM) is swollen in the swelling agent (QM). Subsequently, the polymer (P) is separated from the swelling agent (QM).

The conditions under which the permanent marking (PM) can be distributed in the swelling agent (QM), in particular dissolved or dispersed, the conditions under which the polymer (P) can be swollen in the swelling agent (QM) comprising the permanent marking (PM) and the conditions under which the polymer (P) from the source means (QM) can be separated are dependent on the nature of the polymer (P) and the used swelling agent (QM). These are known to the person skilled in the art and are selected as needed.

One difficulty which may occur by the use of the permanent marking (PM) comprising at least one nucleic acid (NUK) with a known partial sequence (TSEQ) is the low solubility of the nucleic acids in organic solvents which are typically used in the context of polymers and polymerization processes.

In dissolved form, however, the nucleic acids can be incorporated much more homogeneously into the polymer.

One possibility in this connection is the modification of the nucleic acids in order to increase the solubility in organic solvents. For example, it is possible to complex the nucleic acids with organic cations. A corresponding method is described in WO 2013/053483. Suitable nucleic acids and organic cations, as well as a method for modification are described for example on page 9, paragraph 2 to page 12, paragraph 1 of WO 2013/053483.

Another difficulty arises from the tendency of the nucleic acids to decompose and/or degrade. A decomposition or degradation of the nucleic acids is possible, for example, by the action of acids or bases, the action of enzymes and the action of elevated temperatures.

Especially polymerization of the at least one monomer (M) is usually carried out at an elevated temperature.

In one embodiment, the polymerization of the at least one monomer (M) is carried out in a certain temperature range, preferably in a temperature range of 0 to 250° C., more preferably in a temperature range of 20 to 200° C., even more preferably in a temperature range of 25 to 150° C., furthermore, preferably in a temperature range of 30 to 120° C., even more preferably in a temperature range of 35 to 110° C.

Since the nucleic acid (NUK) used in the permanent marking (PM) tends to decompose and/or to degrade also during other process steps, it is preferred that none of the process steps in the process described herein for the authentication of a medical device (MED), respectively in the process described herein for the preparation of a medical device (MED) is carried out at a temperature above 250° C., preferably above 200° C., more preferably above 150° C., furthermore, more preferably above 120° C., in particular above 110° C.

Optionally further process steps may be provided, such as, for example, a process step resulting in crosslinking of the polymer (P).

The term “crosslinking” refers to a reaction in which a large number of individual macromolecules are linked to form a three-dimensional network. This includes both intramolecular crosslinking as well as intermolecular crosslinking. The linkage can be achieved either directly during the polymerization and/or by a polymerization reaction downstream. Methods for the preparation of crosslinked polymers are known to the person skilled in the art and are carried out as required. If “crosslinking” is performed with already formed macromolecules, this may be called a “cross-linking”, which will also fall under the term “crosslinking”. In the polymer sector, the term “curing” or “hardening” is used in this context.

In addition to a change in the mechanical and physical properties of the polymers themselves, crosslinking reduces the tendency of permanent marking (PM) to leave the polymer in an uncontrolled manner. This can be of particular importance, when the polymer (P) for use in the production of the medical device (MED) comprising the permanent marking (PM) is in direct contact with humans when used in humans.

As already explained above, the method for producing the medical device (MED) with a permanent marking (PM) depends on the medical device itself. If an additional process step for crosslinking the polymer (P) is desired, the processes described can be modified accordingly.

In one embodiment, the method may comprise the steps of:
(i) providing at least one monomer (M);
(ii) providing a permanent marking (PM) containing at least one nucleic acid (NUK) with a known partial sequence (TSEQ);
(iii) distributing the permanent marking (PM) in the at least one monomer (M);
(iv) polymerizing the at least one monomer (M) to obtain a polymer (P) comprising the permanent marking (PM);
(v) crosslinking of the polymer (P);
(vi) processing the polymer (P) into a medical device (MED),
   wherein the crosslinking of the polymer (P) can take place during and/or after the polymerization of the at least one monomer (M).

In another embodiment, the method may comprise the steps of:
(i) providing a polymer (P);
(ii) providing a permanent marking (PM);
(iii) distributing the permanent marking (PM) in the polymer (P);
(iv) crosslinking of the polymer (P);
(v) processing the polymer (P) into a medical device (MED).

In this embodiment, it is possible to dissolve the polymer first in order to control the distribution of the permanent marking (PM) and to facilitate the subsequently performed crosslinking.

In this case, the method may include the following steps:
(i) providing a polymer (P);
(ii) providing a solvent (LM);
(iii) dissolving the polymer (P) in the solvent (LM);
(iv) providing a permanent marking (PM);
(v) distributing the permanent marking (PM) in the dissolved polymer (P);
(vii) crosslinking of the polymer (P);
(ix) processing the polymer (P) into a medical device (MED).

Optionally, after the distribution of the permanent marking (PM) in the dissolved polymer (P) and/or cross-linking the polymer (P), the polymer (P) is firstly separated from the solvent (LM) and then crosslinking and/or processing to a medical device (MED) is carried out. In this case, the method for producing a medical device (MED) with a permanent marking (PM) comprises the step of separating the polymer (P) from the solvent (LM).

The conditions under which the process steps of dissolving the polymer (P), distributing the permanent marking (PM) and separating the polymer (P) are carried out depend on the nature of the polymer (P) and the solvent (LM) used. They are known to the person skilled in the art and are selected as needed.

The method for producing the medical device (MED) may thus comprise a cross-linking step, which is carried out during and/or after the polymerization.

In one embodiment, the polymer (P) processed in the medical device (MED) is crosslinked. In this case, the method for producing the medical device (MED) may comprise an additional process step, in which the polymer (P) is crosslinked. This can be carried out during the polymerization of at least one monomer (M) and/or following the polymerization of the polymer (P). Alternatively, in the process for manufacturing a medical device (MED), a polymer which is already crosslinked may be used.

In one embodiment, the crosslinking is carried out in a certain temperature range, preferably in a temperature range of from 0 to 250° C., more preferably in a temperature range of from 20 to 200° C., even more preferably in a temperature range of from 25 to 150° C., still further preferably in a temperature range of from 30 to 120° C., even more preferably in a temperature range of from 35 to 110° C.

The term “polymer (P)” refers to a substance composed of molecules in which one or more types of atoms or atomic groups are repeatedly lined up. In other words, polymers are chemical substances consisting of macromolecules, which are composed of one or more structural units, repeating structural units (monomers). Included are both synthetic and natural polymers, in particular synthetic or natural homopolymers, which are composed of one type of monomer unit, and synthetic or natural copolymers which are composed of two or more different monomer units.

The polymer (P) to be used for the production of the medical device (MED) is not critical as long as it is suitable for the intended use of the medical product (MED) and can be formed under conditions which do not substantially change, in particular fragment the nucleic acid (NUK) having a known partial sequence (TSEQ) contained in the permanent marking (PM).

The term “polymer” in the context of this invention does not refer to a nucleic acid even if a nucleic acid is actually a polymer. In other words, the terms “polymer” and “nucleic acid” are not to be construed equal in the context of this invention. The polymer (P) differs structurally from the nucleic acid (NUK). In particular, the polymer (P) does not comprise nucleosides and nucleotides.

The polymer (P) may be selected, for example, from the group consisting of polyolefins, polyhalogenolefins, styrenic polymers, vinyl polymers, fluoropolymers, polyacrylic and methacrylic polymers, polyoxymethylene, polyamides, aromatic polyesters, aromatic polysulfides, aromatic polyethers, aliphatic polyesters, poly (aryl) ether ketones, aromatic polyimides, polyurethanes, naturally occurring polymers and derivatives, rubbers, aliphatic polyketones and mixtures thereof.

Corresponding polymers are described for example by Saechtling in “Kunststoff Taschenbuch”, 30th Edition, 2007 Carl Hanser Verlag Munich, on pages 445-666.

The polymer (P) may consist of a single type of monomer (M) in that the monomer (M) is reacted in a polymerization reaction to obtain polymer (P). In this case, a homopolymer is obtained. However, it is also possible that the polymer (P) consists of two or more different monomers (M) in that two or more different monomers (M) are reacted in a polymerization reaction to obtain polymer (P). In this case, a copolymer is obtained. Therefore, in addition to the corresponding homopolymers of the above-mentioned polymer classes, corresponding copolymers are also covered.

The nature of the polymer used depends on the desired properties of the medical device (MED) and will be selected by the person skilled in the art accordingly.

In a preferred embodiment, the polymer (P) is a rubber, in particular a silicone rubber and/or silicone rubber derivatives.

The silicone rubber may be selected from the group consisting of polydimethylsiloxane rubber, polydiphenylsiloxane rubber, methyl-phenyl-siloxane rubber, methyl vinyl siloxane rubber, methyl phenyl vinyl siloxane rubber, methyl-fluoro siloxane rubber, corresponding derivatives and mixtures thereof.

In particular, the silicone rubber may be selected from the group consisting of polydimethylsiloxane, polydiphenylsiloxane, polymethylphenylsiloxane, polydimethylsiloxane-co-methylhydrosiloxane, polydimethylsiloxane with terminal vinyl groups, polydiphenylsiloxane with terminal vinyl groups, polymethylphenylsiloxane with terminal vinyl groups, polydimethylsiloxane-co-methylhydrosiloxane with terminal vinyl groups, polydimethylsiloxane with terminal trimethylsilyl groups, polydiphenylsiloxane with terminal trimethylsilyl, polymethylphenylsiloxane with terminal trimethylsilyl, polydimethylsiloxane-co-methylhydrosiloxane with terminal trimethylsilyl and mixtures thereof.

In a preferred embodiment, the polymer (P) is crosslinked and composed of polydimethylsiloxane-co-methylhydrosiloxane with terminal trimethylsilyl and polydimethylsiloxane with terminal vinyl groups. The crosslinking of the polymer (P) takes place via the terminal vinyl groups.

The monomers (M) required for the preparation of the abovementioned polymers (P) are known to the person skilled in the art and are selected as required.

The properties of the medical device (MED), or the properties of the polymer (P), resp., should not be deteriorated by the permanent marking (PM). In order to enhance the compatibility between the permanent mark (PM) and the polymer (P), the permanent marking (PM) may be coated with a polymer (BP) first and then distributed in the monomer (M). A coating of the permanent marking (PM) may also be provided if the permanent marking (PM) is incorporated into the polymer after the polymerization.

The polymer (BP) which is provided for the coating may correspond to the polymer (P) or may be a polymer which is different from the polymer (P). It should be well distributable without affecting the properties of polymer (P).

Typically, such a modified method comprises a step of coating the permanent marking (PM) with the polymer (BP). Only after this coating step the permanent marking (PM) is dispersed in the monomer (M) or polymer (P).

In one embodiment, the method described comprises a step in which the permanent marking (PM) is first coated with the polymer (BP) and is distributed in the polymer (P) after the coating with the polymer (BP) only.

In another embodiment, the method described comprises a step in which the permanent marking (PM) is first coated with the polymer (BP) and is distributed in the monomer (M) after the coating with the polymer (BP) only.

The forgery-proof marking or labeling of the medical device (MED) takes place via a permanent marking (PM), which contains at least one nucleic acid (NUK) with a known partial sequence (TSEQ).

The concept of forgery-proof marking of valuables by the superficial application of nucleic acids is already known.

In contrast, the present method is based not on a subsequently superficially applied marking, but is based on a concept, in which the marking is present in a polymer matrix, which is used for the production of the medical device. In other words, the marking is not subsequently applied, but is already added during the manufacturing process of the medical device and is incorporated into the medical device itself.

The forgery-proof marking of the medical device (MED) takes place via a permanent marking (PM), which contains at least one nucleic acid (NUK) with a known partial sequence (TSEQ). For this purpose, firstly a polymer (P) which is used to produce the medical device (MED) is labeled with the permanent marking (PM) which contains at least one nucleic acid (NUK) with a known partial sequence (TSEQ). The authentication of the medical device (MED) is carried out by isolating and detecting the at least one nucleic acid (NUK) with a known partial sequence (TSEQ), which is contained in the permanent marking (P).

The marking is used in a medical device. It is not desirable due to potential health hazards that the marking leaks from the medical device (MED) uncontrollably, in particular that it bleeds over a longer period.

As already explained above, the permanent marking (PM) is preferably permanently bond to the medical device and cannot be isolated from the medical device (MED) in substantial amounts without any auxiliary means. In other words, the permanent marking (PM) cannot be isolated from the medical device (MED), without changing structurally the device by the auxiliary means or utilities, for example, on dissolution in a suitable solvent, the degradation of the polymer with suitable reagents or mechanical removal by a purposive external influence.

In one embodiment the permanent marking (PM) is not released by the complete contact of the medical device (MED) with TE buffer (10 mM Tris-HCl, 1 mM EDTA, pH 8.0) in an amount above 0.01% by weight-%/h, preferably in an amount exceeding 0.001 by weight %/h, more preferably in an amount of about 0.0001 weight %/h, even more preferably in an amount above 0.00001 by weight %/h from the medical device (MED), based on the total weight of permanent marking (PM).

The selection of the nucleic acid is not critical as long as it contains a characteristic partial sequence which is suitable as a marking carrier. The nucleic acid may be derived from a natural source or may be of synthetic origin. Both a single-stranded and multiple-stranded nucleic acids can be used in the described method. The number of nucleotides is not critical, as long as a partial sequence occurs, which is suitable as a marking carrier and may be detected correspondingly.

The nucleic acid (NUK) with a known partial sequence (TSEQ) is a naturally occurring nucleic acid, a synthetically produced nucleic acid, or a mixture of a naturally occurring nucleic acid and a synthetically produced nucleic acid. The nucleic acid (NUK) having a known partial sequence (TSEQ) may be a deoxyribonucleic acid (DNA) or ribonucleic acid (RNA) or also derivatives, which contain all or a part of rare bases or modified bases. In a preferred embodiment, the nucleic acid (NUK) with a known partial sequence (TSEQ) is a deoxyribonucleic acid (DNA).

In a preferred embodiment, the nucleic acid (NUK) with a known partial sequence (TSEQ) is a synthetically produced nucleic acid.

The nucleic acid (NUK) with a known partial sequence (TSEQ) can be a single-stranded nucleic acid, a double-stranded nucleic acid, or a mixture of a single-stranded nucleic acid and a double stranded nucleic acid.

In a preferred embodiment, the nucleic acid (NUK) with a known partial sequence (TSEQ) is a double-stranded nucleic acid.

In one embodiment, the nucleic acid (NUK) with a known partial sequence (TSEQ) is a double-stranded, synthetically produced nucleic acid having a number of nucleotides in a range from 10 to 10 000, preferably in a range of from 10 to 1000, still more preferably in a range of 15 to 250.

In order to increase the security against counterfeiting or forgery, it is possible to provide, in addition to the nucleic acid (NUK) with a known partial sequence (TSEQ), further nucleic acids which are similar to the nucleic acid (NUK) with a known partial sequence (TSEQ), but do not have the partial sequence required for the authentication (TSEQ) and, therefore, are hardly distinguishable from the nucleic acid (NUK) with the partial sequence (TSEQ) for someone who does not know the partial sequence (TSEQ).

In one embodiment, the permanent marking (PM) comprises, in addition to the nucleic acid (NUK) with a known partial sequence (TSEQ), at least one further nucleic acid which does not have the partial sequence (TSEQ).

This at least one further nucleic acid can be contained in the permanent marking (PM) in a ratio of nucleic acid to nucleic acid (NUK) having a known partial sequence (TSEQ) in the range of from 1/100 to 100/1, preferably 1/50 to 50/1, more preferably 1/10 to 10/1.

In one embodiment, the at least one further nucleic acid is included in at least an equal amount compared to the nucleic acid (NUK) having a known partial sequence (TSEQ) in the permanent marking in order to effectively “hide” partial sequence (TSEQ) which provides authentication. In this case the ratio of nucleic acid to nucleic acid (NUK) having a known partial sequence (TSEQ) can be in the range of 100/1 to 1/1, preferably 50/1 to 1/1, more preferably 1/30 to 1/2, even more preferably 10/1 to 3/1.

The authentication of the medical device (MED) with the permanent marking (PM) takes place via the known partial sequence (TSEQ) of the nucleic acid (NUK) disposed in the permanent marking (PM). For this purpose it is necessary to isolate the permanent marking (PM) from the medical device (MED).

As already explained, the permanent marking (PM) can preferably not be isolated from the medical device (MED) without auxiliary means.

In order to isolate the permanent marking (PM) from the medical device (MED) it is, for example, possible initially to dissolve the medical device (MED) in a suitable solvent and then to isolate the thus released permanent marking (PM). It is also possible that the permanent marking (PM) is removed from the medical device (MED) with a suitable tool by external action.

According to the present invention, isolation of the permanent marking (PM) means that the permanent marking (PM) is made available for the detection of the at least one known partial sequence (TSEQ).

It may be necessary that the permanent marking (PM) has to be searched initially in the medical device (MED). The localization of the permanent marking (PM) in the medical device (MED) may be performed by an additional facilitating feature. For example, it is possible to design the permanent marking (PM) so that it can be optically located.

The nucleic acid (NUK) having a known partial sequence (TSEQ) can initially be treated with a colorant and then dispersed in the polymer (P). Suitable colorants for the gentle treatment of nucleic acids are known to the person skilled in the art and are selected as needed.

The nucleic acid (NUK) having a known partial sequence (TSEQ) can be treated with a colorant. Suitable colorants are known in the art and are selected as needed. For example, N′,N′,-2-trimethylphenothiazine-3,7-diamine chloride can be used as the colorant.

For the authentication of the medical device (MED), the permanent marking (PM) is first isolated with suitable tools. The medical device (MED) is authentic when the at least one known partial sequence (TSEQ) in the at least one nucleic acid (NUK), which is contained in the permanent marking (PM), can be detected.

The person skilled in the art knows suitable methods for detection of the partial sequence of a nucleic acid and will selected them as needed. In this case, such a sequence-specific detection method may comprise a reproduction (amplification) of at least one known partial sequence (TSEQ) in the at least one nucleic acid (NUK). The person skilled in the art knows suitable methods for amplification of nucleic acid and will selected them as needed. Suitable methods for amplification are, for example, the polymerase chain reaction and the isothermal amplification.

In an amplification of nucleic acids, a known partial sequence (TSEQ) of a nucleic acid (NUK) is amplified by means of specific primers. A specific primer oligonucleotide is an oligonucleotide which is extendible by polymerase that specifically hybridizes with a nucleic acid under suitable hybridization conditions. The skilled person will be familiar with suitable methods for selecting a specific primer and will select it as needed. The thus amplified partial sequence of the nucleic acid not only has a specific sequence of nucleotides (sequence) but also a specific number of nucleotides (length).

In order to detect the known partial sequence in a sequence-specific manner, in a first step, the at least one known partial sequence (TSEQ) in the at least one nucleic acid (NUK) can be amplified and in a further step, the length of the amplified nucleic acid can be determined by means of specific primers. The skilled person will be familiar with suitable methods for determining the length of a nucleic acid and will select them as needed. A suitable method for determining the length of a nucleic acid is, for example, gel electrophoresis.

However, depending on the amount of isolated permanent marking (PM) and the methods for detecting the partial sequence of a nucleic acid, the at least one known partial sequence (TSEQ) can be detected also directly and in a sequence-specific manner, i.e. without amplification of the known partial sequence (TSEQ). The person skilled in the art is familiar with suitable methods for directly detecting the partial sequence of a nucleic acid and will select them as required.

Suitable methods for direct detection are, for example, sequencing, i.e. the determination of the nucleotide sequence. Then the sequence thus determined can be compared with the known partial sequence (TSEQ). The skilled person will be familiar with suitable methods for determining the sequence of a nucleic acid and will select them as needed.

A direct detection can also be carried out by hybridization with a nucleic acid which is at least partially complementary to the known partial sequence (TSEQ) of the nucleic acid (NUK).

The nucleic acid for detection may be unlabelled or labeled depending on the method for detecting the partial sequence of a nucleic acid. Suitable nucleic acids for detection are known to those skilled in the art and are selected as needed. For example, the detection of nucleic acid can be a “molecular beacon” which is marked with a fluorescent dye and a quencher. Upon hybridization of a “molecular beacon” with a complementary sequence, the fluorescence properties change. This change in the fluorescence properties can be used as a sequence-specific detection of the known partial sequence of a nucleic acid.

It is known to the person skilled in the art that sequence-specific detection methods or else individual steps of the sequence-specific detection methods can be combined. For example, the at least one known partial sequence (TSEQ) in the at least one nucleic acid (NUK) is amplified in a first step by means of sequence-specific primers and is combined in a further step with one of the direct detection methods described above.

The detection of the known partial sequence can be carried out qualitatively and/or quantitatively. If a quantitative detection is carried out, then the method according to the invention permits not only qualitative authentication of the medical device (MED) but also quantitative authentication, i.e. the determination as to whether the polymer (P) comprising the permanent marking (PM) has been blended or diluted with a structurally similar or identical polymer. In other words, it is possible with the inventive method to carry out a quantitative authentication of the medical device (MED) and thus to determine via the polymer (P) which has been marked with the permanent marking (PM), whether it has been “diluted” with a structurally similar or identical polymer.

Suitable quantitative sequence-specific detection methods are known to the person skilled in the art and are selected as required. For example, the detection may include a quantitative polymerase chain reaction. A quantitative detection of the known partial sequence can comprise, for example, the use of molecular beacons, as the change in fluorescence properties caused by the hybridization permits not only a qualitative, but also a quantitative detection.

For a quantitative authentication of the medical device (MED), it is first necessary to distribute the permanent marking (PM) as homogeneously as possible in the polymer (P), taking into account a certain tolerance range. At a later point in time, the medical device (MED) is qualitatively and quantitatively authenticated via the nucleic acid (NUK) contained in the permanent marking with a known partial sequence (TSEQ). In this case, the concentration of the permanent marking (PM) in the polymer (P) is determined, and is compared with the concentration of permanent marking (PM) in polymer (P) being used for the manufacture of the medical device (MED). If the deviation is within the tolerance range, it can be assumed that the polymer (P) was not mixed or diluted with a structurally similar or identical polymer.

In one embodiment, the method for authentication of the medical device (MED) additionally comprises the following steps:
(i) determining the concentration (EK) of the permanent marking (PM) in the polymer (P);
(ii) comparing the concentration (EK) of the permanent marking with the concentration (AK) of the permanent marking (PM) of the polymer (P) which has been used to prepare the medical device (MED).

In one embodiment, a defined volume (V) of the polymer (P) is used for the determination of concentrations (EK) and (AK), for example, a volume (V) of 0.01 to 30 cm³, preferably a volume of 0.05 to 10 cm³, more preferably a volume of 0.1 to 3 cm³.

In one embodiment, the tolerance range in the determination of the concentrations (EK) and (AK) comprises a deviation of up to 5 wt.-%, preferably a deviation of up to 2 wt.-%, more preferably a deviation of up to 1 wt.-%, even more preferably a deviation of up to 0.5 wt.-%, even more preferably a deviation of 0.05 wt.-%, based on the total weight of the permanent marking (PM) in the volume (V).

A medical device (MED) with the permanent marking (PM) comprising a polymer (P) with a permanent marking (PM) is also described wherein the permanent marking (PM) contains at least one nucleic acid (NUK) (with a known partial sequence TSEQ).

What was said in connection with the procedure for authenticating the medical device (MED) via the polymer (P), the marking (PM), the nucleic acid (NUK) and the partial sequence (TSEQ) also applies to the medical device (MED) as such.

Also described is the use of a permanent marking (PM) comprising at least one nucleic acid (NUK) with a known partial sequence (TSEQ) for authenticating a medical device (MED).

The statement made in the procedure for authenticating the medical device (MED) via the marking (PM), the nucleic acid (NUK) and the partial sequence (TSEQ) also applies to the use of a permanent marking (PM) for authenticating a medical device (MED).

### EXAMPLES

The following describes the authentication of a silicone rubber. Silicone rubber can be used for the manufacture of a medical device, for example, a breast implant shell.

In order to authenticate the silicone rubber genomic DNA (gDNA) of a tomato was incorporated into a liquid silicone mixture. The gDNA was permanently embedded in the silicone rubber by a platinum catalyzed cross-linking reaction. The isolation of the permanent gDNA labeling was carried out with the aid of a scalpel. Two known partial sequences of the gDNA were detected by polymerase chain reaction (PCR) and subsequent gel electrophoresis.

### Preparation of the Silicone Rubber with Permanent Marker

A crosslinkable two-component liquid silicone rubber system (Silbione® LSR 4305 HC from Bluestra Silicones crosslinked over a Pt catalyst) was used. The LSR rubber system contained polydimethylsiloxane having terminal vinyl groups and polydimethylsiloxane-co-polymethylhydrosiloxane with trimethylsilyl groups, each of which were used as a dispersion in pyrogene hydrophobic silica.

As the permanent marking a gDNA from tomatoes was used. For this purpose, gDNA was isolated from 2 g of tomato leaves (young plants, variety Tomimaru muchoo). The isolated gDNA was dissolved in 500 μl of TE buffer (10 mM Tris, 1 mM EDTA). 10 μl of this solution served as a reference for later detection (Sample 1). After the concentration was determined, the sample was precipitated and dried in vacuum.

361±40.6 μg of dried gDNA were incorporated into the liquid silicone mixture. For this purpose, the two components of the LSR rubber system, each 1 g, were mixed and centrifuged. The dried DNA pellet was carefully placed between two layers of the liquid silicone mixture so that it was completely enclosed from the liquid silicone mixture. Subsequently, the liquid silicone mixture was cured under vacuum for 8 h at 105° C.

### Determination of the Barrier Properties of the Silicone Rubber

The gDNA pellet embedded in the silicone rubber was roughly cut out of the material under the microscope. It was ensured that the gDNA remained completely enclosed by the silicone rubber.

The excised gDNA pellet was completely covered with about 1 ml TE buffer. After 4 days, the TE buffer was assayed for released DNA molecules. For this purpose, 10 μl of TE buffer were taken (sample 2), and have been examined by PCR and subsequent gel electrophoresis.

### Isolation of the Permanent Label

The silicone rubber-sample with the enclosed gDNA pellet was removed from the TE buffer and gDNA pieces have been excises using a scalpel under the electron microscope. This piece of gDNA (sample 3) was amplified by PCR and subsequent gel electrophoresis.

### Detection of a Known Partial Sequence of the gDNA

A PCR was performed with the three different gDNA samples (Sample 1, Sample 2 and Sample 3).

Each sample was amplified with two different primer pairs:
Primer pair 1: 5′-TACTGGTGGTTTTGAAGCTG-3′ (SEQ ID no.: 1), 5′-AACTTCCTTCACGATTTCATCATA-3′ (SEQ ID no.: 2); expected length of amplified DNA: 279 base pairs (bp);
Primer pair 2: 5′-AGACCACGAGAACGATATTTGC-3′ (SEQ ID no.: 3); 5′-TTCTTGCCTTTTCATATCCAGACA-3′ (SEQ ID no.: 4); expected length of the amplified DNA: 92 base pairs (bp)

Subsequently, the amplified DNA sections of the PCR samples were analyzed by means of a 2% agarose gel.

By comparison with a 50 bp marker it was found that the PCR of the reference sample (sample 1: gDNA before inclusion in the silicone rubber) with the primer pair 1 resulted in an amplified DNA segment in the size range from 250 to 300 bp. With primer pair 2, a DNA segment in the size range of 90 to 110 bp was amplified. The sizes of the amplified DNA portions of Sample 1 were the same as those of Sample 3 (DNA cut out of the silicone rubber with the scalpel). Thus, during curing at 105° C. for 8 hours, the gDNA was trapped undamaged in the silicone gum and the known partial sequence could be detected after isolation of the DNA from the silicone rubber.

In the sample 2 (TE buffer which was in contact with the fully enclosed DNA marking for 4 days) no DNA segments were amplified. This suggests that the trapped gDNA could not be released from the silicone matrix.

### Influence of the Crosslinking Temperature

For this purpose, the gDNA pellets were used which have been dyed before drying in vacuo with a 0.02% to 0.12% toluidine blue solution (N′,N′-2-trimethylphenothiazine-3,7-diaminchlorid).

The dyed DNA pellets were embedded in the silicone rubber as described above. The crosslinking was carried out at different temperatures: 105° C. for 8 h, 150° C. for 6 h, 175° C. for 5 h and 200° C. for 4 h.

When crosslinked at 105° C. and crosslinked at 150° C. h the dyeing of the gDNA pellets remained unchanged. The enclosed gDNA was then cut using a scalpel. Known partial sequences of the excised gDNA could be detected as described above by means PCR and subsequent gel electrophoresis.

When crosslinked at 175° C. and crosslinked at 200° C. the dyeing of the gDNA pellets changed. Black colored brownish gDNA was isolated, but known partial sequences could no longer be detected in the excised gDNA.

### Determining the Proportion of Non-Crosslinked Silicone Rubber

For implants, free silicone (non-crosslinked silicone) can cause damage to the patient's body. Therefore, the uncrosslinked silicone content was investigated as a function of the crosslinking temperature.

The determination was performed by means of a Soxhlet extraction using n-hexane. The non-crosslinked silicone (free silicone) was dissolved or extracted from the silicone matrix by the solvent n-hexane. The insoluble, cross-linked silicone swelled up in n-hexane and remained in the extraction sleeve.

The highest degree of crosslinking in the silicone matrix was achieved at the lowest vulcanization temperature in conjunction with the longest vulcanization time (105° C., 8 h). With a sample quantity of 0.55 g, the non-crosslinked silicone content was 23.8%. The lowest degree of crosslinking in the silicone matrix and thus the highest non-crosslinked silicone content, which was 29.1%, was obtained under a vulcanization condition of 200° C. for 4 h. At 150° C. for 6 h the non-crosslinked silicone content was 27.3%, at 175° C. for 4 h 29.0 %.

1. A method of authenticating a medical device (MED), comprising the following steps:
(i) producing a medical device (MED) having a permanent marking (PM), wherein the medical device (MED) comprises a polymer (P) with the permanent marking (PM) and the permanent marking (PM) contains at least one nucleic acid (NUK) with a known partial sequence (TSEQ);
(ii) isolating the permanent marking (PM); and
(iii) authentication of the medical device (MED) by detection of the at least one known partial sequence (TSEQ).

2. The method according to claim 1, wherein the proof comprises:
(i) the amplification of at least one known partial sequence (TSEQ) of the at least one nucleic acid (NUK), and/or
(ii) sequencing of at least one known partial sequence (TSEQ) of the at least one nucleic acid (NUK), and/or
(iii) the hybridization of the at least one known partial sequence (TSEQ) of the at least one nucleic acid (NUK) with a detection nucleic acid.

3. The method according to claim 1, wherein the authentication of the medical device (MED) by detection of the at least one known partial sequence (TSEQ) comprises the following steps:
(i) determining the concentration (EK) of the permanent marking (PM) in the polymer (P) of the medical device (MED) to be authenticated;
(ii) comparing the concentration (EK) of the permanent marking with the concentration (AK) of the permanent marking (PM) of the polymer (P) used to make the medical device (MED).

4. Method of producing a medical device (MED) with a permanent marking (PM) comprising the following steps:
(i) providing a polymer (P) with a permanent marking (PM), wherein the permanent marking (PM) contains at least one nucleic acid (NUK) with at least one known partial sequence (TSEQ); and
(ii) processing the polymer (P) into a medical device (MED).

5. The method according to claim 4, wherein the production of the medical device (MED) comprising the following steps of:
(a1) providing at least one monomer (M);
(a2) providing a permanent marking (PM) containing at least one nucleic acid (NUK) with a known partial sequence (TSEQ);
(a3) distributing the permanent marking (PM) in the at least one monomer (M);
(a4) polymerizing the at least one monomer (M) to obtain a polymer (P) comprising the permanent marking (PM); and
(a5) processing the polymer (P) into a medical device (MED), or
(b1) providing a polymer (P);
(b2) providing a permanent marking (PM);
(b3) distributing the permanent marking (PM) in the polymer (P); and
(b4) processing the polymer (P) into a medical device (MED), or
(c1) providing a polymer (P);
(c2) providing a solvent (LM);
(c3) dissolving the polymer (P) in the solvent (LM);
(c4) providing a permanent marking (PM);
(c5) distributing the permanent marking (PM) in the dissolved polymer (P); and
(c6) processing the polymer (P) into a medical device (MED).

6. The method according to claim 1, wherein the polymer (P) contained in the medical device is crosslinked.

7. The method according to claim 1, wherein the nucleic acid (NUK) having a known partial sequence (TSEQ) is a deoxyribonucleic acid (DNA) and/or wherein the medical device (MED) is a medical device of class III.

8. The method according to claim 1, wherein the permanent marking (PM) in addition to the nucleic acid (NUK) having a known partial sequence (TSEQ) contains at least one further nucleic acid, which does not have the partial sequence (TSEQ).

9. The medical device (MED) with a permanent marking (PM), comprising a polymer (P) with a permanent marking (PM), wherein the permanent marking (PM) contains at least one nucleic acid (NUK) having a known partial sequence (TSEQ).

10. (canceled)


SEQUENCE LISTING (4 sequences)

SEQ ID NO: 1 - DNA, 20 bp, Solanum lycopersicum
tactggtggttttgaagctg

SEQ ID NO: 2 - DNA, 24 bp, Solanum lycopersicum
aacttccttcacgatttcatcata

SEQ ID NO: 3 - DNA, 22 bp, Solanum lycopersicum
agaccacgagaacgatatttgc

SEQ ID NO: 4 - DNA, 24 bp, Solanum lycopersicum
ttcttgccttttcatatccagaca